(19) United States
(12) Patent Application Publication
(10) Pub. No.: US 2016/0278688 A1
(43) Pub. Date: Sep. 29, 2016

(54) Method and System for Diagnosing Uterine Contraction Levels Using Image Analysis

(71) Applicant: FERRING B.V., Hoofddorp (NL)

(72) Inventors: Piotr PIERZYNSKI, Bialystok (PL); Waldemar KUCZYNSKI, Bialystok (PL)

(21) Appl. No.: 14/442,620

(22) PCT Filed: Nov. 26, 2013
(86) PCT No.: PCT/EP2013/074712

§ 371 Date: May 13, 2015

Related U.S. Application Data
(60) Provisional application No. 61/729,870, filed on Nov. 26, 2012.

(51) Int. Cl. A61B 5/00; G06T 7/00; G06T 11/20; A61B 8/08; A61B 17/435

(52) U.S. Cl. CPC A61B 5/4356; A61B 8/08; A61B 8/5223; A61B 17/435; G06T 11/206; G06T 7/0089; A61B 2576/02; G06T 2210/41; G06T 2207/30044; G06T 2207/20116; G06T 2207/10132

(57) ABSTRACT

Method of analyzing uterine contractions by analyzing uterine images using deformable model networks in support of embryo transfer techniques. The method is also used to diagnose premature uterine contractile activity in mammals. The method can be used to control contractile activity during embryo transfer or premature labor when used in conjunction with oxytocin antagonists.

### BACKGROUND

Detection of uterine contractile activity is necessary in a number of medical procedures, including embryo transfer during in vitro fertilization, and well as detecting and preventing preterm labor during pregnancy.

Elevated uterine contractile activity in women undergoing embryo transfer (“ET”) may affect ET success rates. In ET recipients with “silent” uteri, successful implantation rates may be as much as 3 fold higher as compared to patients with elevated uterine contractile activity. It has recently been hypothesized that the application of oxytocin antagonists may decrease uterine contractions and may improve pregnancy rates. In implantation research trials carried out on mice, it was confirmed that oxytocin antagonists reverse the negative effect of oxytocin.

In humans, ethical issues do not permit the use of invasive techniques for the assessment of uterine contractions, such as intrauterine pressure measurement, on patients who are about to undergo embryo transfer procedure. Even if those ethical issues did not exist, the use of any invasive method of measurement as a precursor to an embryo transfer procedure is not advisable and consequently, indirect and non-invasive methods for assessment of contractions must be used. Transvaginal sonography with assessment of endometrial interface movements has been presented in literature in various contexts, and several methods have been devised in an attempt to resolve the issue of contraction assessment. Sonography, generally an ultrasound-based diagnostic imaging technique, is used for visualizing subcutaneous body structures. Typical diagnostic sonographic scanners operate in the frequency range of 2 to 18 megahertz, though frequencies up to 50-100 megahertz have been used experimentally in a technique known as biomicroscopy in special regions, such as the anterior chamber of the eye. The choice of frequency is a trade-off between spatial resolution of the image and imaging depth: lower frequencies produce less resolution but image deeper into the body. Higher frequency sound waves have a smaller wavelength and thus are capable of reflecting or scattering from smaller structures. Higher frequency sound waves also have a larger attenuation coefficient and thus are more readily absorbed in tissue, limiting the depth of penetration of the sound wave into the body.

Sonography (ultrasonography) is widely used in medicine. It is possible to perform both diagnosis and therapeutic procedures, using ultrasound to guide interventional procedures (for instance biopsies or drainage of fluid collections). Sonography is effective for imaging soft tissues of the body. Superficial structures such as muscles, tendons, testes, breast and the neonatal brain are imaged at a higher frequency (7-18 MHz), which provides better axial and lateral resolution. Deeper structures such as liver and kidney are imaged at a lower frequency 1-6 MHz with lower axial and lateral resolution but greater penetration.

In 1998, R. Fanchin published an article in Human Reproduction 1998:13(7):1968 proposing a method based on analyzing the cross-section of a line segment and video sequence that creates a two-dimensional plot using successive frames; the horizontal component representing line segment length and the vertical component representing time. Although simple and easy to implement, the method clearly demonstrates drawbacks. In the presence of a slight increase in the amount of noise or movement of the whole organ, the method is prone to generate incorrect results or results that do not provide useful information relating to contractility. These drawbacks are the product of not incorporating a model in the process and by using only low-level image data in the analysis. The method has been tested but because of the wide variety of real input that occurs, it was not found to be an accurate tool for the measurement of contractility.

Model-free techniques, such as that referred to above, comprise a large number of methods and are amongst the oldest used in image analysis. The feature that distinguishes all of them is that they only use low-level image data and thus do not profit from the a priori assumptions relating to object shape and location. Thus, their application is limited by a number of conditions specific to medical imaging. Some such methods, e.g. thresholding, even neglect to use the information provided by the spatial location of pixels—a key factor in accurate image processing frameworks—preferring instead to use only their numerical value. Model free techniques include use of amplitude mode (A-mode), brightness mode (B-mode) and motion mode (M-mode) sonography.

M-mode sonography creates an image of an organ by emitting ultrasound pulses in quick succession, typically using either an A-mode or a B-mode image with each pulse. Over time, and linking multiple successive images together, the boundaries and velocities of the moving organ can be determined. A disadvantage of the M-mode method in detecting uterine contractions is that it does not provide the means to segment the whole uterus, only the upper and lower boundaries present within a user specified intersection. This lack of provision becomes significant in cases of exaggerated bowel or respiratory movement that may change the location of previously marked gaps in the uterine boundary. Additionally, the uterus can move forward or backward in relation to the intersection that has been set. Such movement might seem to be irrelevant but it can result in producing a segmental plot that is indistinguishable from a contraction. Further problems manifest themselves in the form of noise or false edges. Since the method does not take the whole uterine shape into account, only the boundaries crossing the intersection are trackable and this can make measurement difficult. Even in ideal conditions where the boundaries are clear and easy to track, accurate measurement can still be difficult to accomplish. Similarly, it is also difficult to interpret images where, although contractility is present, the movement of boundaries remains static with only a textural change of the endometrium being influenced. The technique is also dependent on proper visualization of the uterus which is highly variable and influenced by factors such as its retrovert position or filling of the urinary bladder.

### SUMMARY

It is an object of the present invention to provide a system and method for detecting uterine contractions. It is a further object of the present invention to detect uterine contractions using deformable model networks during embryo transfer procedures. It is a further object of the present invention to detect uterine contraction in the early stages of preterm labor during pregnancy.

In an embodiment of the present invention, a method includes gathering ultrasound images of a subject uterus over a period of time, analyzing the images using a deformable model network to identify uterine contractions, and displaying uterine contractions in a graphical format. In a further embodiment of the present invention, uterine contractions are determined to be within a minimum or maximum threshold in terms of intensity or frequency. The frequency of the contractions can be between 0 and 15 contractions per minute.

In still a further embodiment of the present invention a method is provided for delivery and transfer of an embryo to a uterus comprising: collecting of one or more eggs from a subject patient; providing luteal support to the patient using for example micronized progesterone; fertilizing the one or more eggs to provide a viable embryo; qualifying uterine contractions in the patient by recording images of the uterine contractions and evaluating the images using a deformable models network; reducing the level of contractions to under 4 contractions per minute; transferring the embryo to the uterus; continuing luteal support by administering e.g. micronized progesterone.

In yet another embodiment of the present invention, a method of analyzing uterine images comprises: recording uterine images over a period of time; setting reference axes for use in a deformable model network; setting the outer snake surrounding the endometrium of the subject uterus; setting the inner snake within the endometrium of the subject uterus; applying one or more image filters to enhance one or more features of interest; relaxing the snakes (the snakes move to the points by taking a minimum energy measure of possible points in a neighborhood surrounding each point) until both meet at the endometrium perimeter; displaying the recording and snake movement on a user display. Parameters of inner and outer snakes (such as rigidity, elasticity, number of axes and others) are predefined for the average ultrasound image so the snakes are best outlining the endometrium; they can also be custom modified by the user. Snake movement is supervised by an observer live on the screen during the analysis, in case of any noise (such as sudden movement of a patient resulting an unexpected change in image parameters), introducing bias in snake positioning, the analysis can be halted, and the axes and active contours (snakes) can be re-set.

In a further embodiment of the present invention a method is provided to detect uterine contractions using deformable model networks in women in pre-term labor, the method comprises: gathering ultrasound images of a subject uterus over a period of time, analyzing the images using a deformable model network to identify uterine contractions, and displaying uterine contractions in a graphical format, determining if the measured uterine contractions are within a minimum or maximum threshold in terms of intensity or frequency, wherein the frequency of the contractions can be between 0 and 15 contractions per minute.

In still a further embodiment of the present invention, a method is provided to detect and stop pre-term labor contractions, the method comprising: gathering ultrasound images of a subject uterus over a period of time, analyzing the images using a deformable model network to identify uterine contractions, and displaying uterine contractions in a graphical format, determining if the measured uterine contractions are within a minimum or maximum threshold in terms of intensity or frequency, wherein the frequency of the contractions can be between 0 and 15 contractions per minute; and administering an oxytocin antagonist. The oxytocin antagonist can be any oxytocin antagonist, such as, but not limited to atosiban or barusiban. Atosiban can be administered in one or more doses. Atosiban can be administered in three doses. Atosiban can be administered in a first injection of 0.9 ml intravenous bolus over one minute with a dose of 6.75 mg, in a second injection of 24 ml/hour over three hours of intravenous loading at a dose of 18 mg/hour, and a third injection via intravenous infusion of 8 ml/hour at a dose of 6 mg/hour.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is an M-mode recording a uterine transection.

FIG. 2 is a deformable models network based recording

FIG. 3 is an M-mode recording a uterine transection.

FIG. 4 is a deformable models network based recording.

FIG. 5 is an M-mode recording.

FIG. 6 is a deformable models network based recording.

FIG. 7 is a comparison of an IUP recording and a CPP recording.

FIG. 8 is an M-mode recording.

FIG. 9 is a deformable models network based recording.

FIG. 10 is an M-mode recording.

FIG. 11 is a deformable models network based recording.

FIG. 12 is an M-mode recording.

FIG. 13 is deformable models network based recording.

FIG. 14 is an intrauterine pressure recording.

FIG. 15 is an M-mode recording.

FIG. 16 is a deformable models network based recording.

FIG. 17 is an M-mode recording.

FIG. 18 is a deformable models network based recording.

FIG. 19 is an intrauterine pressure recording.

FIG. 20 is an M-mode recording.

FIG. 21 is a deformable models network based recording.

FIG. 22 is an M-mode recording.

FIG. 23 is a deformable models network based recording.

FIG. 24 is an intrauterine pressure recording.

FIG. 25 is an M-mode recording.

FIG. 26 a deformable models network based recording.

FIG. 27 is an M-Mode recording.

FIG. 28 is a deformable models network based recording.

FIG. 29 is an intrauterine pressure recording.

FIG. 30 is an M-Mode recording.

FIG. 31 a deformable models network based recording.

FIG. 32 is an intrauterine pressure recording.

Like reference symbols in the various drawings indicate like elements.

### DETAILED DESCRIPTION

Uterine contractile activity, one of the key components of uterine receptivity has been shown to influence pregnancy rates in assisted reproductive therapy (ART) patients. It has been demonstrated that oxytocin/vasopressin VIA antagonists promote implantation in an animal model. In human embryo transplant recipients, such treatment is expected to decrease contractions and improve the pregnancy rates.

Embryo Transfer (ET) procedure is an independent factor affecting the success rates of IVF-ET treatment. To be effective, ideally it should be non-invasive. This is especially important in view of the fact that the hyperestrogenic uterine environment is thought to promote the expression of myometrial oxytocin receptors and therefore, potentially increases sensitivity to oxytocin and other contractors. It has been demonstrated that stressor stimuli such as tenaculum used during the embryo transfer, increases contractions for up to 60 minutes (Lesny P, Human Reproduction 1998; 13(6):1540.). It has also been shown that cervical insertion and dilatation may evoke uterine contractions (Handler J et al., Theriogenology 2003; 59:1381.). Consequently, any invasive procedures including intrauterine pressure assessment should be avoided before and during embryo transfer. There is a need for an effective tool for non-invasive measurement of uterine contractions before and during ET, that enables the assessment of potential medication.

Uterine contractions have previously been monitored by M-mode measurements techniques, as described above. Although being non-invasive, M-mode measurements techniques have several limitations. Such limitations include: sensitivity to different sizes of uteri and endometrial thickness, image noise, breathing movements, and so forth.

Implementations of the present invention use a deformable models network in a method of image analysis that can be applied to the same film sequences used in M-mode measurement, resulting in more accurate data. In one implementation of the present invention, the computer based deformable models network application provides results that are more robust, noise-resistant and more consistent than those using M-mode assessment. The method provides data on overall changes of image structure in the whole of the sagittal transection, not just a single image segment (as in M-mode assessment) or single point (as in intrauterine pressure assessment). Consequently, using a deformable models network provides more global and more accurate measurements over previous techniques.

In an implementation of the invention, the computer based deformable models network application also enables raw data from the graph representing uterine contractions to be used for further processing and analysis. Using deformable models networks also eliminates outliers automatically, and is much less sensitive to technical instability of the image. Implementations of the present invention provide relative values and the result is not dependent on uterine diameters or magnification of the image.

Deformable model approaches to uterine imaging, such as a computer based deformable models network application also delineates amplitude of contractions. Statistical processing of signals also allows calculations of the area under curve to reflect the strength of contractions. Differences in profile between intrauterine pressure (IUP) recordings and Snake Studio measurements can be attributed to the fact that IUP is measured at a single point of the uterus as opposed to the global assessment provided by Snake Studio. IUP is dependent not only on strength of myometrial contractions, but also on intra-abdominal pressure, breathing movements, positioning of the catheter, and finally, the state and thickness of the endometrium. Consequently, it is not possible to directly compare intrauterine pressure changes recorded using IUP with those measured by a computer based deformable models network application (i.e. discrete texture changes of endometrium may be connected to pronounced IUP changes, or the reverse). The deformable models network method however provides recordings that may be considered superior to IUP, insofar that it provides more global data.

Using volunteer patients and employing simultaneous IUP and ultrasound assessments, made it possible to compare both types of recording. It has been shown that the computer based deformable models network application is highly consistent as compared to intrauterine pressure. It may be especially useful in cases when M-mode provides inconsistent or inconclusive data. It has been demonstrated that this method provides advantages to M-mode recordings.

For example, deformable models networks provide data on overall changes of image structure in the whole sagittal trans-section, resulting in more global and accurate measurements over M-mode method analysis, which does not provide the means to segment the whole uterus, only the upper and lower boundaries present with in a user specified intersection. Also deformable models provide measurements related to the whole organ and are less sensitive to variable magnifications in sonography, whereas M-mode recordings are size sensitive (i.e. the absolute amplitude will depend in image size). Deformable models do not require any manipulation on the recorded material, whereas M-mode recordings can require conversion and manipulation of the film sequences. Also M-mode recordings do not allow for the exclusion of artifacts in the same manner that deformable models do.

Additionally, raw data from the graphical representation of the uterine contraction supports further processing and analysis in deformable model networks, but M-mode methods do not allow further analysis from the graphical data. Deformable models also allow for the calculation of statistics delineating uterine contractile activity; are not sensitive to body movement and other image instability, are more independent of the visualization of the uterus, and are less sensitive to signal noise.

To overcome the drawbacks of M-mode presentation based packages of uterine contraction monitoring, implementations of the present invention utilize a comprehensive method of imaging based on a deformable objects framework which generates a greatly enhanced and more useful output. Deformable models (also called “snakes”) were introduced in 1988, (See, Kass M., Witkin A., and Terzopooulos, International Journal of Computer Vision; 1988; 1(4):321).

Deformable models have become a powerful method for image analysis with several variants in use. Such images are characterized by a great variety of extracted objects e.g. noise, artifacts due to the acquisition process, inconsistent object boundaries, spatial luminance changes, etc. Deformable models are capable of reducing the impact of these corruptions to provide more robust and accurate segmentation. This often allows manual segmentation to be eliminated which, as a process is laborious, unrepeatable and—due to the presence of human-based errors—often unreliable. Although the human factor is still necessary to supervise the process, most of the aforementioned issues are overcome using deformable models. The other area that greatly benefits from deformable objects is motion tracking; the model can be naturally expanded to accommodate shape changes in time.

This new method is a compilation of a framework called “United Snakes” that was first proposed by Liang, McInerey and Terzopolous in Medical Image Analysis in 2006 (Liang et al., Medical Image Analysis 2006; 10(2):215-233) and a method called “Dual Active Contour” proposed by Gunn and Nixon in 1997 (Gunn SR and Nixon MS, IEEE Transactions on Pattern Analysis and Machine Intelligence Archive 1997; 19(1): 63). The method is fine-tuned and uses a set of image filtering tools to cope with the specific problems that acquired video sequences exhibit. Moreover, it is capable of extracting a wide spectrum of objects from various images and video sequences.

In implementations of the present invention, two-dimensional deformable models are represented by closed curves. The initial two snakes are placed in the image by the operator, one outside the object (the endometrium) that is to be extracted, and the other within it. There is no need to place the initial snakes near the boundaries, the only constraint being that the snakes cannot cross them. Opposing forces are applied that make the snakes move toward each other, following which they are allowed to deform under other specific forces. One force is referred to as intern and its purpose is to preserve a required shape. By adjusting this force the operator can make the snakes perform like a rigid rod, or like a soft rope, or any degree of malleability between these two extremes. The Second force is referred to as “external”, and this determines how the snakes are attracted by image data (e.g. luminance changes). The snakes deform under the forces specified to reach the lowest possible energy level that fits the image thus allowing a required shape to be preserved.

Segmentation is performed with a priori information about the object that is to be extracted, something that is passed over by most other segmentation methods. Basically, the snake behaves in a manner similar to that of the human brain. The brain has a general idea of the location and shape of an object, which it then transforms into a specific image by tailoring the model to the image data available. Some areas of the object overlap with luminance changes and are accepted whereas others are ignored if it would result in a shape that is considered unacceptable. Snake segmentation can be considered as a very similar process. A priori model is embedded in the image and works in unison with low-level data to produce an accurate result. Moreover, a high order of constraints exists that determine the output characteristics of the object. For example, the snake may be set up to form a rigid object that would be less affected by noise and other artifacts or which otherwise might be capable of fitting image data more accurately.

After identifying the object a set of statistics is computed. The snakes are then moved away (for a predetermined distance) and again relaxed on the subsequent frame (some frames can be skipped if continuous frames only slightly differ).

Uterine contractile activity is assessed using a combination of statistics that reflect dynamic changes in endometrial shape and image texture, including changes taking place during the contractions. Contractility Presence Probability (CPP) values may range between 0 (lack of uterine contraction) and 1000 (simulated uterine contraction based on mathematical model).

The statistics module included within the computer based deformable models network application endeavors to match a set of predefined statistics with a “model/ideal contractility pattern”, which is considered to reflect how the shape (especially the thickness of the uterine along the model) and the texture (whether it flows locally or is equally distributed) changes at different stages of the contraction. If the statistics follow exactly the model contraction along the whole timeline the video scores 100 (never happens), for the constant shape and texture—the score is 0. There is post processing step to eliminate outliners and “average the statistics” within a small time frame (to eliminate small frame-to-frame inconsistencies).

The method is fast enough to perform in real time and is relatively simple to interpret. It also has the potential to label different types of contractility which, in itself, is of considerable value. To make the method easier to use as a uterine contractility tool, a profile of default settings can be created leaving only initialization of the snakes to the user, which is straightforward and not more complicated than the initialization of the method based on M-mode ultrasound. Another important advantage is that the computer based deformable models network application provides a much greater level of output data which simplifies interpretation and presents a far more detailed picture of uterine contractile activity; a factor that is of significant importance in instances where uterine contractile activity causes changes in image texture without effecting the shape of the endometrium.

The application uses Microsoft DirectX technology to access video memory and process recorded video frames prior to them being rendered on screen. The work environment that the application offers is both customizable and flexible, and consists of modules through which various operations can be performed:
Format Properties—shows information about the video file format and performance statistics
Playback Properties—give access to the playback rate and size options
Preprocessing Filters—allows various graphical filters to be applied to the image
Snake Properties—gives access to the snake parameters
Snake Coordinates—displays coordinates of the snake's nodes
Timeline Analysis—provides a means to mark intervals of interest on the video timeline
Timeline Plot—displays how the snake statistics vary through the time.

These modules allow almost all aspects of segmentation to be controlled independently and can be used to filter the image and tune the snake to work with new types of videos.

Analysis is performed in real time and is visualized by a statistics plot that is generated on the fly. The application also offers many other features e.g. video window scrolling, single frame step, frame capture, controlling the alpha channel of the control information and the ability to display cursor position in video coordinates.

Redundancy caused by relatively slight difference between continuous frames may be avoided by specifying the rate at which the snake's position is recomputed. This enables the production of graphical data that reflect disturbances in the endometrial image representing uterine contractions.

The method is projected to be applied as a semi-diagnostic tool offering fast access to results and which may be used for the determination of uterine contractile activity and the need for medication.

### Examples

To validate the computer based deformable models network application method, a clinical study was proposed to provide cross verification of the method against measurements of intrauterine pressure. The study involved patients who underwent controlled ovarian stimulation and volunteered for mock embryo transfer (mock ET) and assessment of intrauterine pressure. All volunteers had undergone controlled ovarian stimulation and had mock ETs. Although initially, stimulation cycles in patients included were planned as therapeutic cycles, in all cases proceeding further with the treatment was not possible due to exaggerated ovarian response or fertilization failures. After consenting to the procedure, the patients received standard luteal support (200 mg tid of micronized progesterone vaginally). Mock ETs and ultrasound scans were performed 2 days after oocyte collection or 2 days+36 hours after hCG administration in whom oocyte collections were not commenced. The assessments in two menstruating volunteers were commenced for the verification of suitability of deformable models network in cases with relatively thin endometrium.

The following methodology was employed. Every patient had a sonography film sequence of sagittal uterine transection recorded prior to undertaking IUP measurements. Next, after removing the vaginal probe and positioning a speculum with side access, an outer sheet of Labotect Embryo Transfer Catheter loaded with Micro Tip pressure catheter SPC 330 (Millar Instruments, US) was introduced into the uterus and positioned in the uterine isthmus according to transabdominal scan. Subsequently, after fixing the ET catheter, the speculum was carefully removed and the transvaginal probe was again carefully introduced into the vagina. Flexibility of the outer sheath of Labotect catheter and intrauterine pressure catheter enabled us to carefully remove the speculum and to again introduce the ultrasound vaginal probe. After confirming positioning of the catheter, measurement of intrauterine pressure was initiated simultaneously with sonography scan recording.

By using a fine and flexible intrauterine pressure catheter, the whole procedure was similar to a mock ET. The Tip of the ET catheter was positioned just behind the internal cervical os and the IUP catheter was introduced inside the uterus for 1.5 cm, but without touching the fundus as this by itself might have invoked contractions and biased the recording. Overall, the whole time of intrauterine pressure measurements was limited to less than 10 minutes. It has not been associated to any significant discomfort to patients, however due to a potential risk of intrauterine infection, a prophylactic course of 5-days of doxycycline (100 mg bid) was prescribed after the transfer. All patients gave their written consent for the procedure before processing. No unwanted effects were observed.

The following equipment was utilized:

Intrauterine Pressure Measurements:
Micro Tip Catheters type SPC 330—flexible polyurethane catheter approved for human use, French size 3 (0.9 mm), pressure sensor mounted at tip (Millar Instruments Inc., US).
Embryo Transfer Catheters—(Labotect GmbH, Germany)
Power Lab 2000 Data Acquisition System (Millar Instruments Inc., US).
Chart 5 for Windows Data acquisition software (ADInstruments, US)
PC computer

Scans
Aloka SSD 1700 scanner with 7.5 Mhz sector vaginal 2d probe
Sony video camera
Pinnacle Studio video processing package

Analysis of Sonography Film Sequences
PC computer station for data acquisition
Snake Studio package for assessment of uterine contractions
M-mode measurements package (specially created operational package producing M-mode graphs of uterine contractions)

Format of Results
Measurements of intrauterine pressure—Chart v 5.5.9 graphs
M-mode assessments of uterine contractions—graphs illustrating movements of endometrial interface
Deformable models network assessments of uterine contractions—graphs illustrating changes of Contractility Presence Probability (CPP) in time

Results:

Intrauterine pressure recordings were compared to recordings of CPP recorded by Snake Studio and M mode recordings. Results for each patient are presented separately

Patient SS01

Age: 25

Fertility Profile in the early follicular phase: FSH 7.4 IU/ml; LH 5.5 E2 25.9 pg/ml; PRL 59 ng/ml; T 0.38 ng/ml

Stimulation protocol: short protocol with buserelin, Clomiphene citrate (50 mg for 5 days) and Fostimon (50 IU every other day—3 doses given)

Ovarian response: 2 follicles 16-18 mm present in the ovary on the day of triggering

Uterine response: Endometrial thickness 9 mm

Concentration of estradiol at the end of COS: 296 pg/ml

Additional data; cycle cancelled after 12 days of ovarian stimulation for IVF, after consenting for the IUP measurements, patient received 10.000 IU of hCG and started micronized progesterone until the day of IUP measurement (2 days+36 hours after triggering)

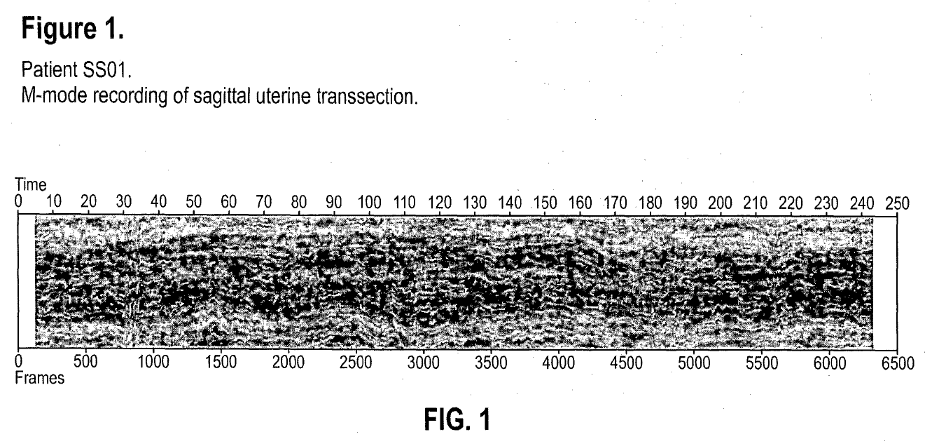

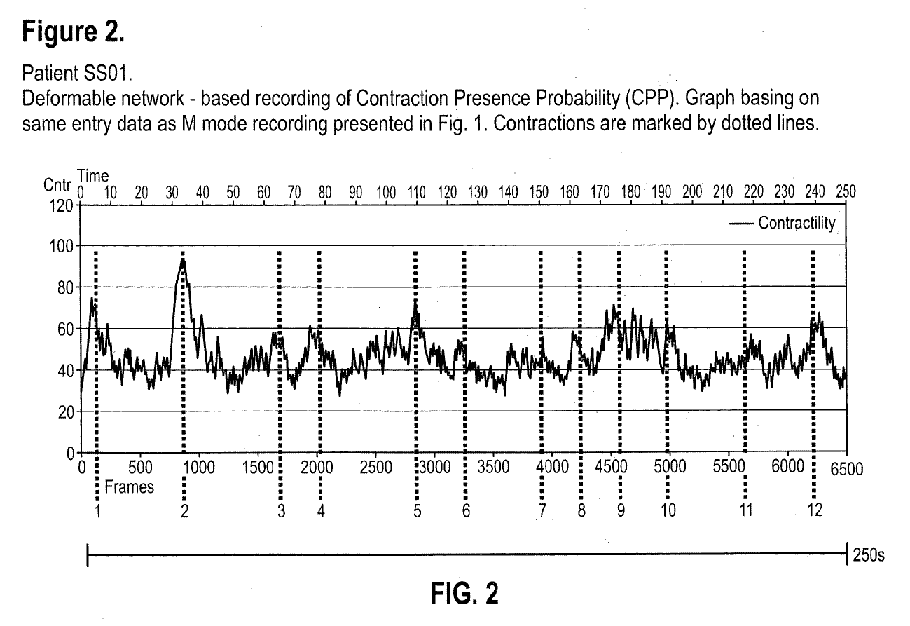

FIG. 1 illustrates the M-mode recording of sagittal uterine transection. FIG. 2 illustrates the deformable models network-based recording of Contraction Presence Probability (CPP)—a measure calculated by the software, which represents uterine contractions.

In M mode method, no clear-cut contractions can be identified (FIG. 1).

Recording employing the identical entry data (the same ultrasound film sequence) when analyzed by deformable models network allows identification of a total of 12 contractions, identified by peaks at approximately times 4, 33, 62, 75, 110, 150, 160, 175, 190, 220, 230, and 240 (FIG. 2).

In this patient, the intrauterine pressure catheter was positioned suboptimally which did not allow to have a satisfying quality of the recording and no pressure measurement are available.

M mode measurements are actually not showing changes which can be attributed to contractions or being visibly different from noise. Snake Studio measurements provided good quality signal and measurements which could be used for counting the number of contractions. Moreover, in contrast to M mode results, the Snake Studio data are formatted in numeric values and can be used for statistical analysis. M mode provides a method for producing ultrasound images which made possible to quantify the number of contractions, however, an output is a graphical file which needs to be a subject of further, laborious analysis.

Patient SS02

Age: 29

Fertility Profile in the early follicular phase: FSH 9.8 IU/l; LH 3.6 IU/l; E2 65.1 pg/ml; PRL 29 ng/ml; T 0.43 ng/ml.

Stimulation protocol: Short protocol with buserelin; COS: Fostimon 150 IU/d for 5 days+Menopur 150 IU/d for 3 days

Ovarian response: 10 mature follicles

Uterine response: endometrial thickness 11 mm

Lab measures at the end of COS: estradiol 2807 pg/ml; PGS 0.81 ng/ml

Comment: Poor oocyte quality, failure to fertilize in all oocytes after ICSI, patient consented to IUP measurements after oocyte collection, patient received 10.000 IU of hCG and started micronized progesterone until the day of IUP measurement (2 days after oocyte collection)

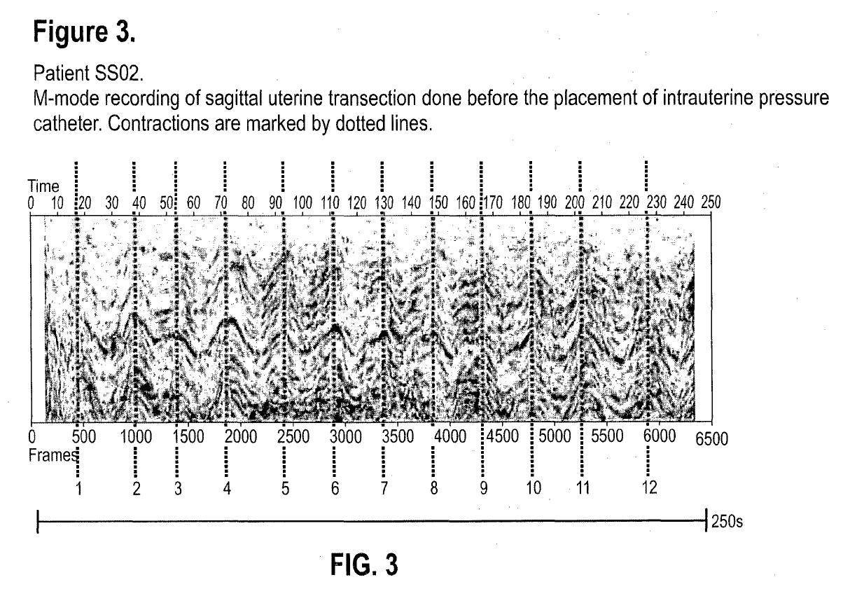

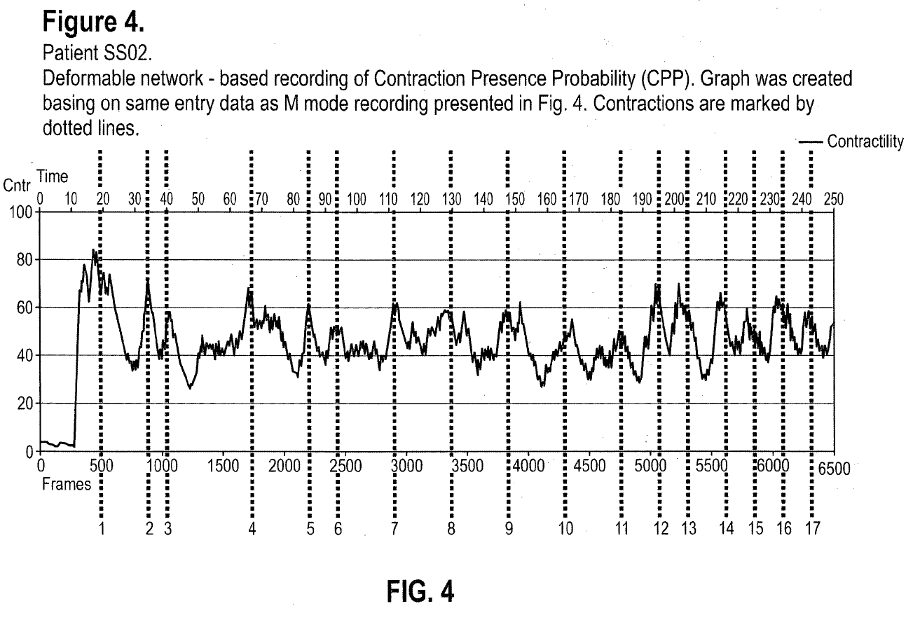

FIG. 3 illustrates the M-mode recording of sagittal uterine transection taken before the placement of intrauterine catheter (mock embryo transfer). On that graph it was possible to identify 12 contractions. FIG. 4 illustrates the same signal analysed using deformable models network. Contraction Presence Probability (CPP) measurements used the identical entry data as M method allowed more accurate identification of contractions as compared to M mode method—a total of 18 contractions was confirmed on this recording (as opposed to 12 contractions as presented on FIG. 3).

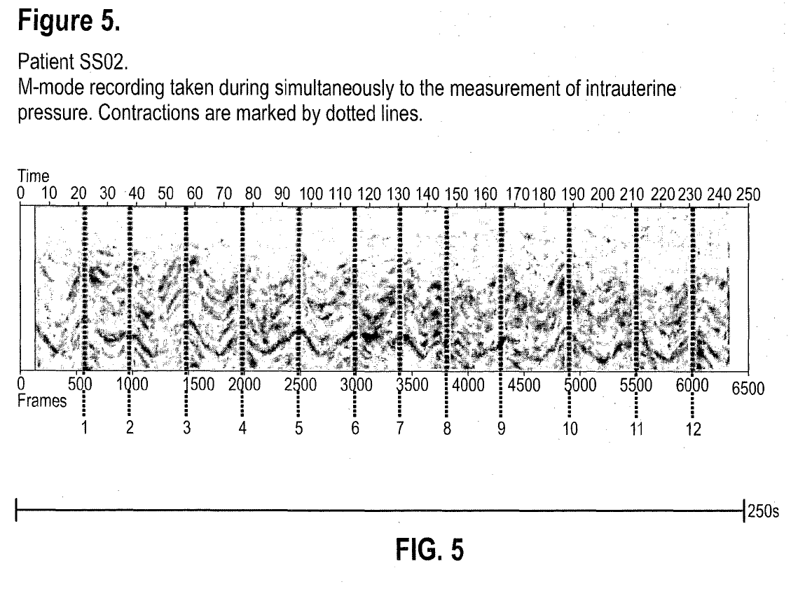

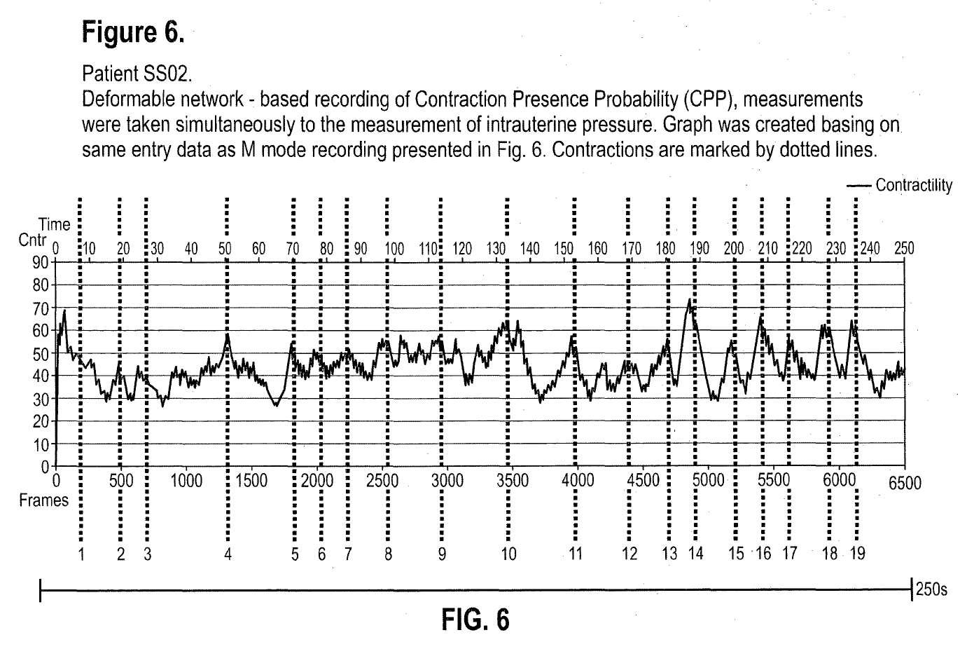

Directly after the recording described above, an intrauterine catheter was inserted through patient's uterine cervix and placed within the uterine cavity. Simultaneous ultrasound scan recording and intrauterine pressure recording were re-started. FIG. 5 illustrates the M-mode recording taken at the time of measurement of intrauterine pressure. FIG. 6 illustrates the CPP recording produced by deformable network-based method using the identical entry data as the M mode recording (shown in FIG. 5).

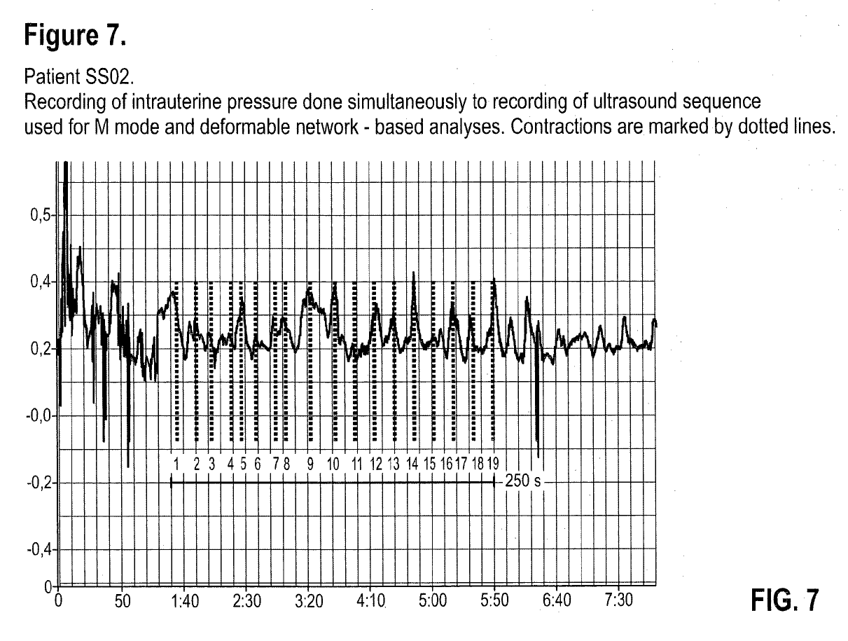

FIG. 7 illustrates the recording of intrauterine pressure which was simultaneous to the recording of the ultrasound scan (analysis of that shown in FIGS. 5 and 6). Intrauterine pressure recordings were taken simultaneously to ultrasound scan, this being enabled by using a flexible Labotect embryo transfer catheter as an outer sheath for IUP catheter. Appropriate positioning of IUP catheter was verified on the scan. In the Intrauterine Pressure Recording, within the analyzed segment of 250 seconds, a total of 19 contractions were identified (FIG. 7). Using the Snake Studio, the same number of contractions was identified on ultrasound recording (FIG. 6). In turn, M mode detected 12 contractions (FIG. 5). The example shows that results produced by Snake Studio were more accurate as compared to M Mode method.

Intrauterine pressure values and values of CPP (produced by deformable models network) are in a form of a raw data file, which allows their further analysis. In the results of the M mode recording, an image presenting the movements of endometrial interface is produced. Extracting numerical data from such an image is complicated and subjective. Additionally, considering that deformable models network provides data delineating the changes in the whole area of sagittal transection of endometrium, it may also be considered as being at least as reliable as the reference recording of intrauterine pressure which—although providing very reliable data, it only does its measurements at a single point of uterus.

Patient SS03

Age: 31

Fertility Profile in the early follicular phase: FSH 11.6 IU/ml; LH 3.0 IU/ml; E2 27.2 pg/ml; PRL 17.2 ng/ml; T 0.47 ng/ml.

Stimulation protocol: short flare protocol with Diphereline (0.1 mg/day, starting on CD1)+150 IU Fostimon on CD 2-10

Ovarian response: 4 mature follicles

Uterine response: Endometrial thickness 12 mm

Concentration of estradiol at the end of COS: 576 pg/ml

Additional data: initially planned for IUI, cycle cancelled due to risk of multiple pregnancy. After consenting to IUP measurements, hCG 10.000 was administered, IUP measurements were taken 4 days later, patient used barrier contraception until the end of cycle, and no complications were noted.

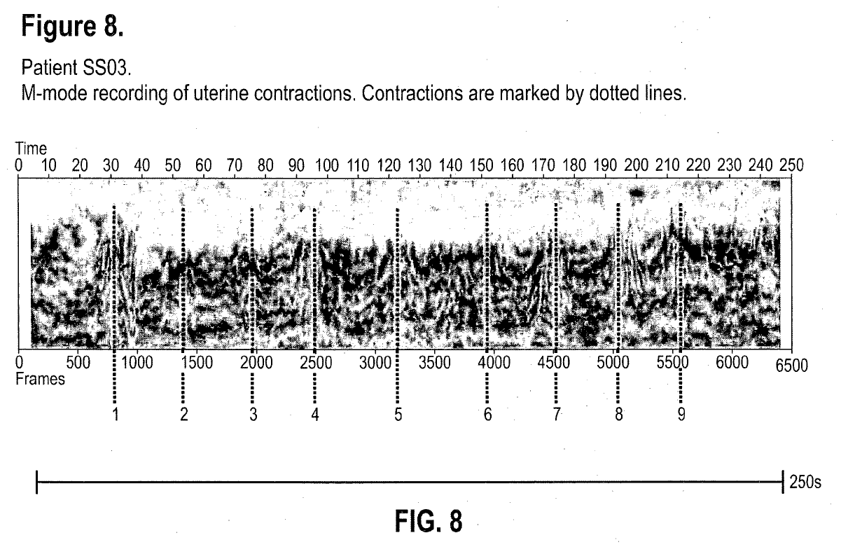

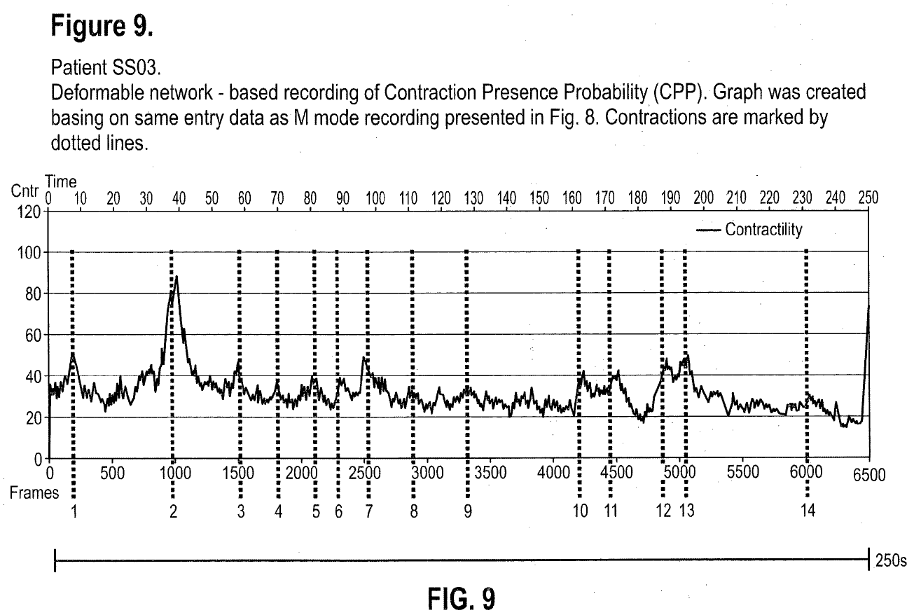

FIG. 8 illustrates the M-mode recording of uterine contractile activity. FIG. 9 illustrates the deformable network-based recording of changes in image parameters based on the same study as M mode recording presented on FIG. 8.

In this patient, simultaneous recording of intrauterine pressure did not provide conclusive readings due to accidental disconnection of intrauterine pressure. Uterine contractions are easily identified on the Snake Studio recording and on the M-mode recording. Snake studio produced more complex recording, providing more information on uterinecontractile activity in this patient.

Patient SS04

Age: 25

Fertility profile in the early follicular phase: FSH 4.9 IU/ml; LH 2.2 IU/ml; E2 53.4 pg/ml; PRL 25 ng/ml; T 0.44 ng/ml

Stimulation protocol: short flare protocol with 0.1 mg diphereline/day+150 IU Fostimon from CD3 to 8

Ovarian response: 21 mature follicles

Uterine response: normal

Concentration of estradiol at the end of COS: E2>3000 pg/ml (exact concentration not measured due to evident clinical picture); PGS 1.1 ng/ml

Comment: 12 oocytes retrieved, patient decided not to undergo the ET in this cycle (embryos were frozen). IUP measurements were conducted 2 days after the oocyte collection. 5000 IU of hCG were administered 36 hours before the oocyte collection, standard luteal support was given, and measurements were taken 2 days after the oocyte collection.

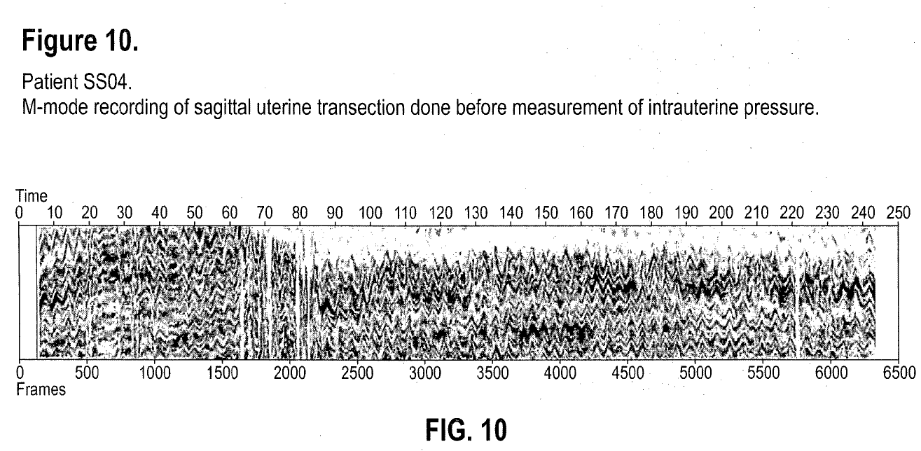

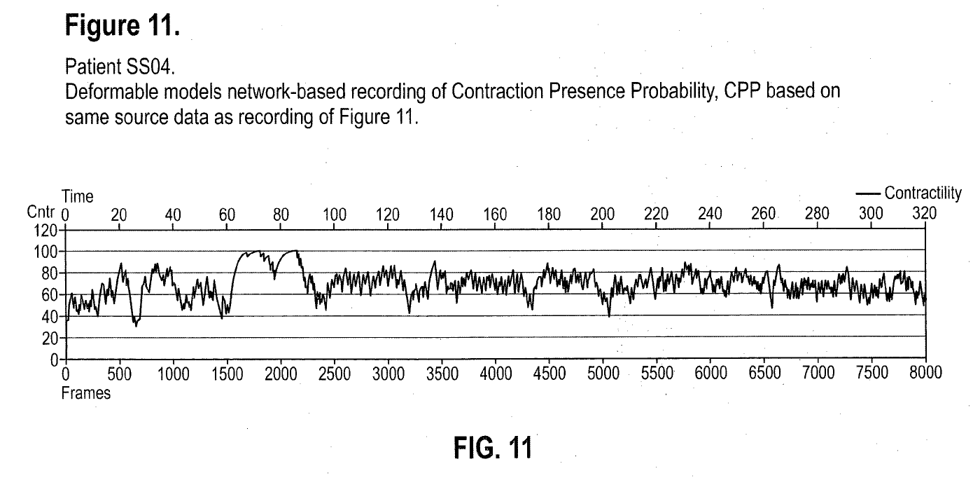

FIG. 10 illustrates the M-mode recording of sagittal uterine transection performed before the measurement of intrauterine pressure. FIG. 11 shows the deformable models network based recording of Contraction Presence Probability, CPP based on the same film sequence as M mode recording of FIG. 10.

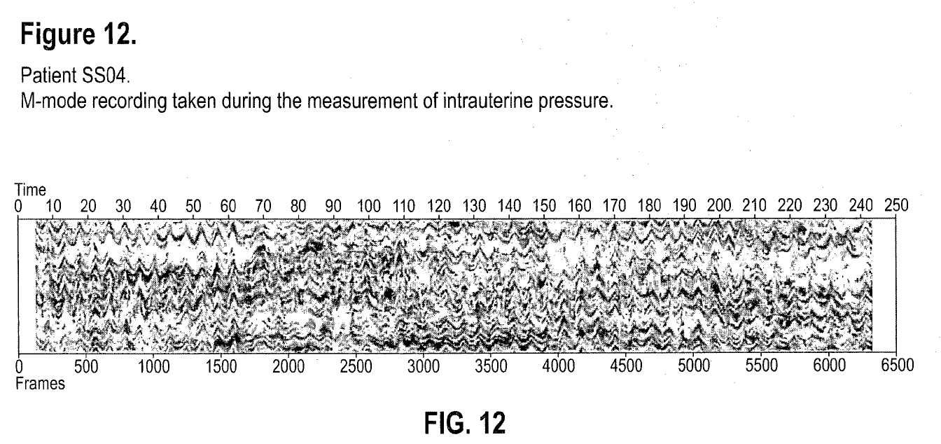

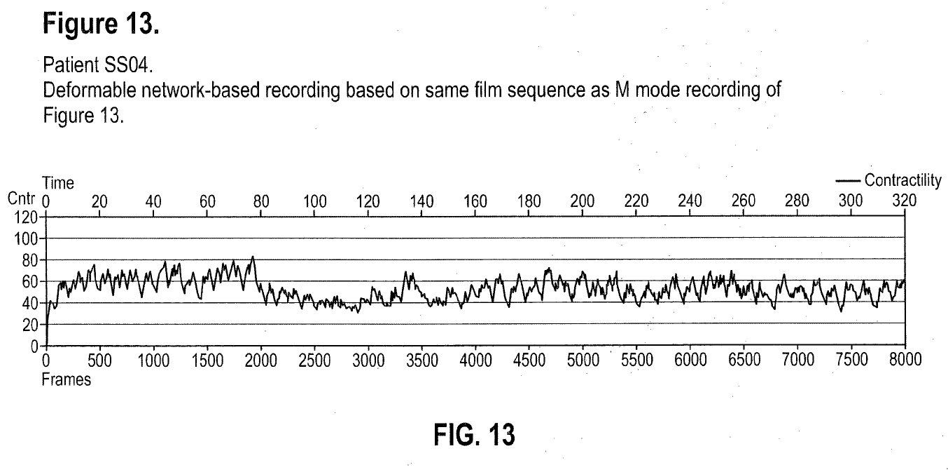

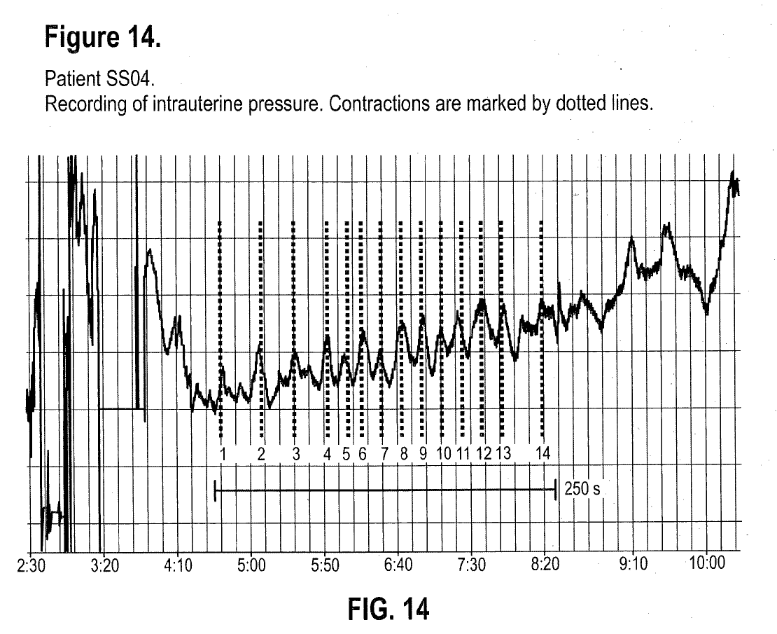

FIG. 12 presents the M-mode recording taken during the measurement of intrauterine pressure. FIG. 13 illustrates the deformable network-based analysis based on the same film sequence as M mode recording of FIG. 13.
FIG. 14 shows recording of intrauterine pressure. Quality of M-mode recording was significantly affected by patient's breathing movements. Snake Studio recordings are more resistant to noise and are more readable than M-mode recordings. Additionally, the Snake Studio recordings are similar to IUP measurements, appropriately reflecting uterine contractile activity.

Patient SS05

Age: 21

Fertility Profile in the early follicular phase: FSH 12.0 IU/l; LH 4.4 IU/l; E2 78 pg/ml; PRL 41.9 ng/ml; T 0.64 ng/ml.

Stimulation protocol: short flare protocol with buserelin, 150 IU of Fostimon for 10 days (CD 3-13)

Ovarian response: two mature follicles

Uterine response: endometrial thickness 13 mm

Concentration of estradiol at the end of COS—291 pg/ml, PGS—1.36 ng/ml

Comment: Cycle abandoned due to insufficient ovarian response, good endometrial picture, identifiable uterine contractions, patient volunteered to IUP measurement after Pregnyl administration.

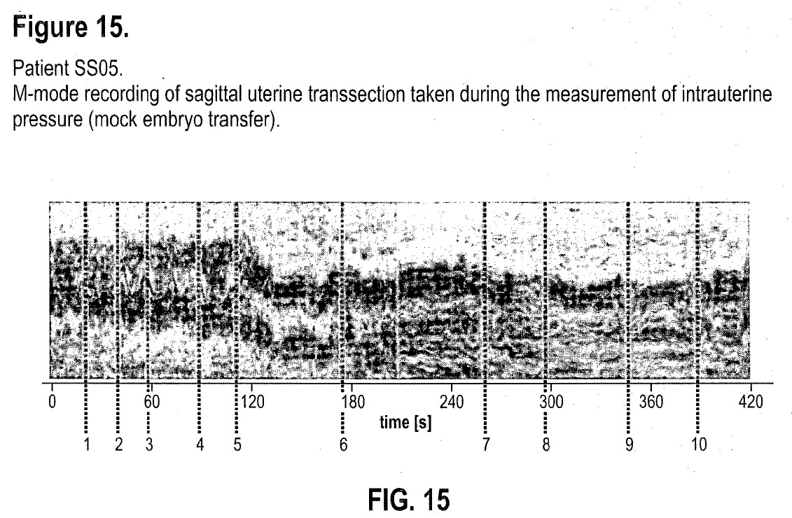

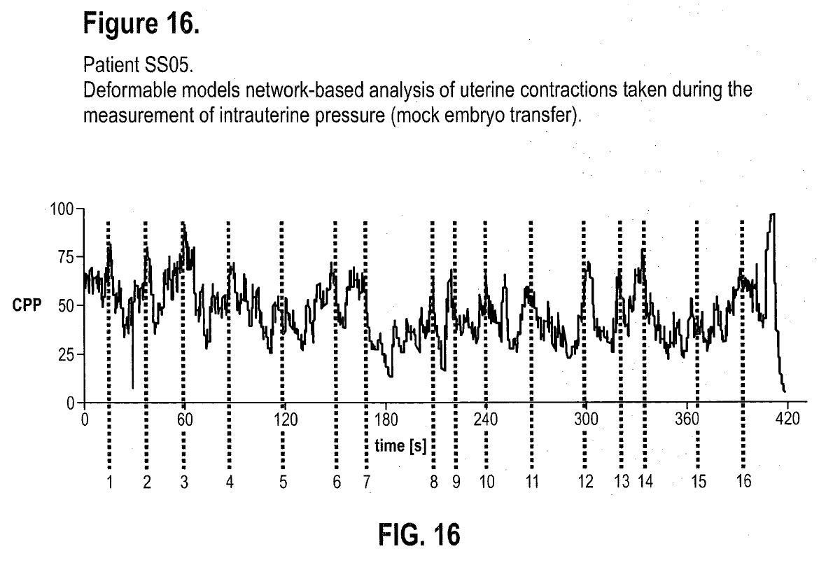

FIG. 15 is an M mode recording of sagittal uterine transection and Snake Studio recording taken during mock embryo transfer. Intrauterine pressure recording did not commence due to a technical fault with the IUP catheter. Ultrasound recording is about 7 minutes duration and for technical reasons, the M-mode graph must have been separated into two parts (note the vertical break line in the 180s-240s segment). M-mode recording allowed indentifying a total of 10 contractions whilst deformable models based method identified 16 contractions. Such a figure was in concordance to observation of film sequence of the ultrasound scan (that was used for both M-mode and deformable network based evaluation of contractions) which detected 15 contractions. The recording done by Deformable models network-based method is presented at FIG. 16.

Patient SS06

Age: 43

Volunteer patient during her menstrual period had IUP measured simultaneously to ultrasound image recording.

Additional data: Uterine contractions were identifiable while inspecting the ultrasound recording. IUP recording demonstrated intensive uterine contractile activity.

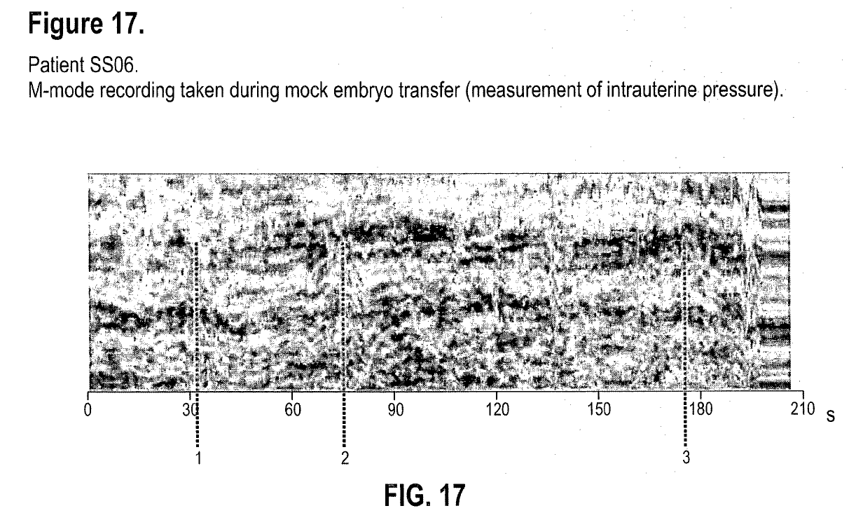

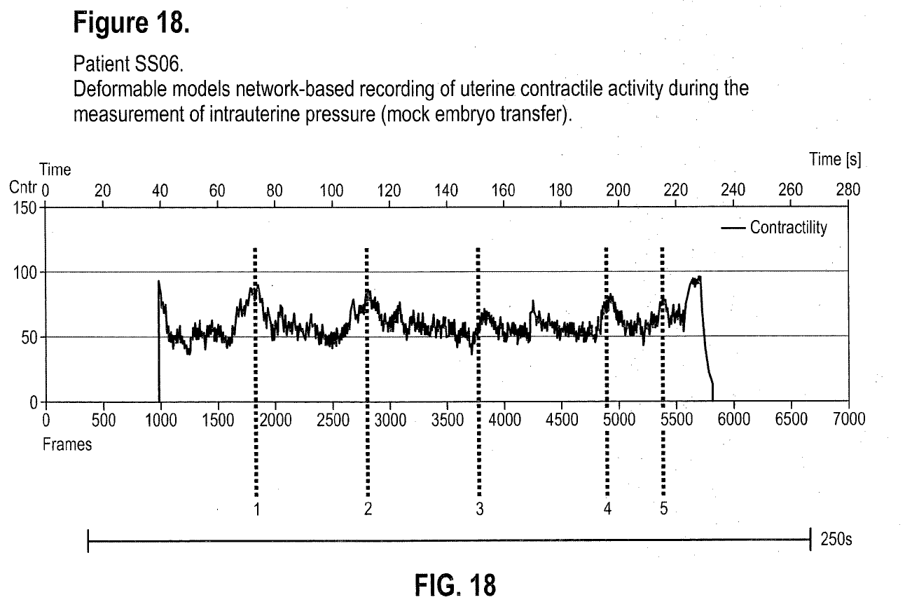

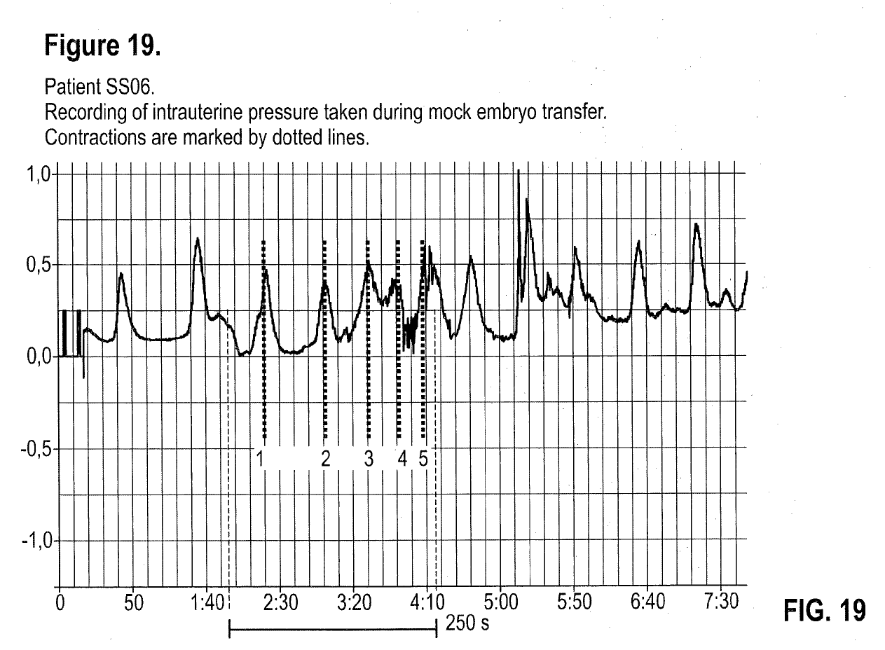

FIG. 17 illustrates M-mode recoding taken simultaneously to measurements of intrauterine pressure. It allowed to identify 3 contractions. It is of note that visualization of contractions was rather complicated in this case, probably due to thin endometrium. FIG. 18 presents recording of uterine contractile activity evaluated by deformable models network-based method. It allowed identifying 5 contractions, which was in concordance to intrauterine pressure measurements presented in FIG. 19. Application of deformable models network allowed accuracy of identification of contractions which was comparable to the reference—invasive—method of intrauterine pressure. By contrast, the M-mode recording produced an inconclusive result. Conversely, Snake Studio demonstrated its ability to provide significant data on uterine contractions even when based on poor quality images (thin endometrium).

Patient SS07

Age: 28

Fertility Profile in the early follicular phase: FSH 4.4 IU/ml; LH 2.8 IU/ml; E2 32.7 pg/ml; PRL 48 ng/ml; T 0:41 ng/ml

Stimulation protocol: Short flare protocol with buserelin; Fostimon 150 IU/d for 5 days+Menopur 150 IU/d for 3 days

Ovarian response: 10 mature follicles, significant risk of HOSS

Endometrial response: good, endometrial thickness 11 mm

Concentration of estradiol at the end of COS: 4243 pg/ml; PGS 1.24 ng/ml

Comment: ET not done due to risk of OHSS. IUP measurements taken 2 days after the oocyte collection, 5 COCs collected, 2 reached blastocyst phase and were cryopreserved. Standard luteal support administered until the IUP measurements.

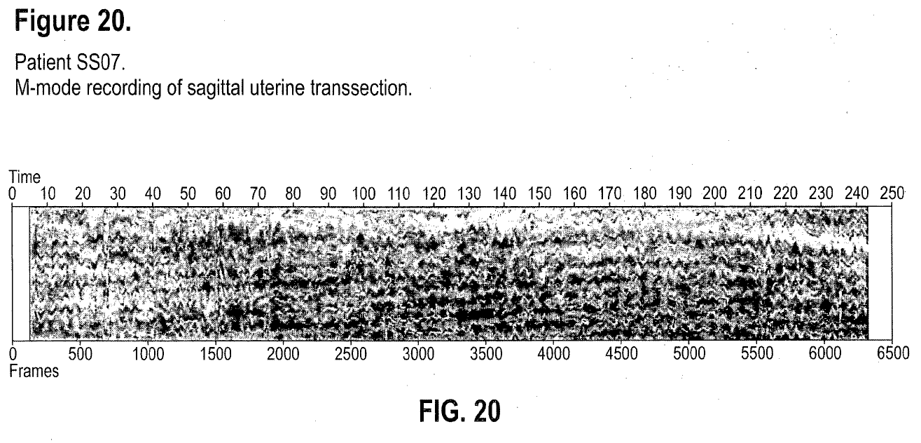

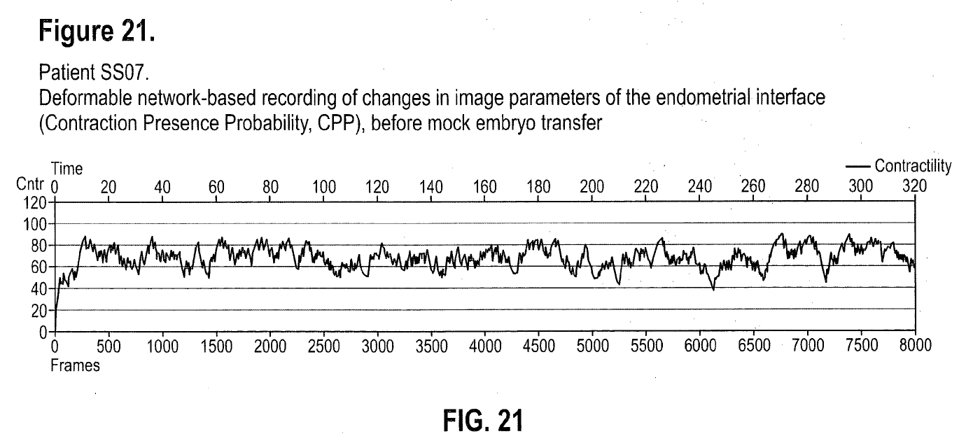

FIG. 20 illustrates the M-mode recording of sagittal uterine transection taken before the measurement of intrauterine pressure (mock embryo transfer). Patient's breathing movements resulting in rather noisy “signal” on ultrasound. Consequently, in M mode measurement presented in FIG. 20 no contractions could be identified. FIG. 21 illustrates the deformable models network based recording of changes in image parameters of the endometrial interface (Contraction Presence Probability, CPP)—measurements taken on the same source data as presented in FIG. 20. In this analysis, uterine contractile activity can distinctively seen.

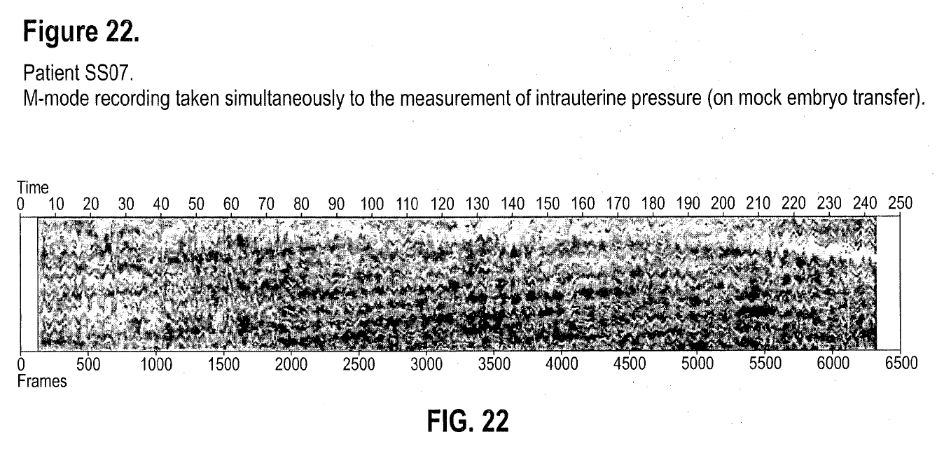

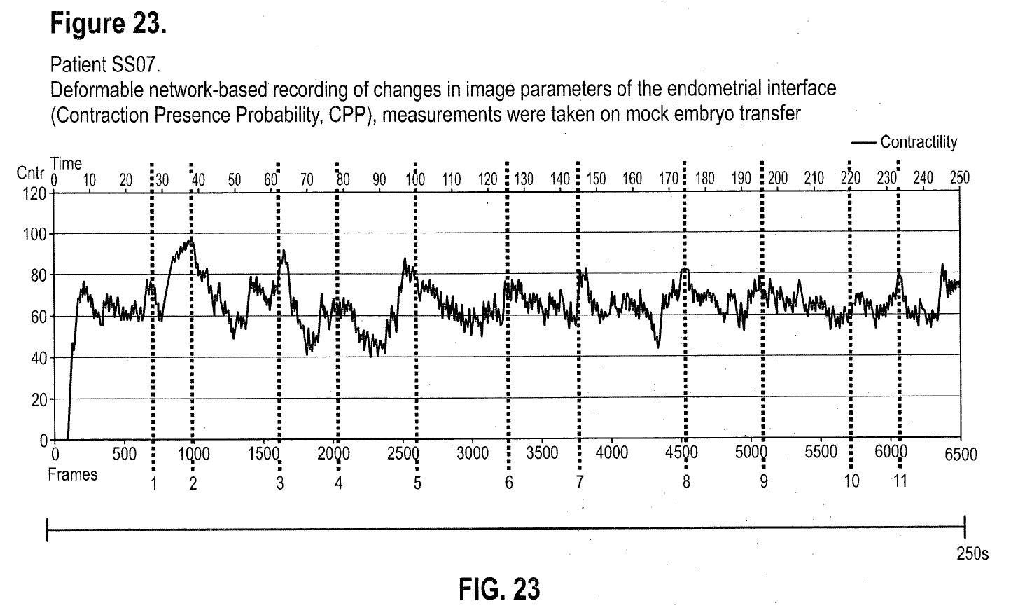

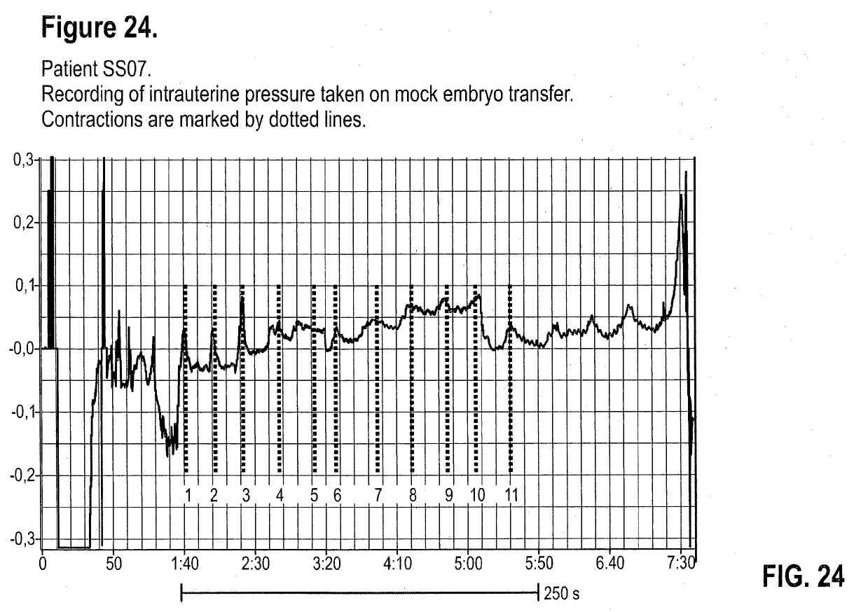

FIG. 22 illustrates the M-mode recording taken during the measurement of intrauterine pressure (mock embryo transfer). Due to high level of noise (breathing movements), no contractions could be identified.
FIG. 23 illustrates the deformable network-based recording taken simultaneously to the measurement of intrauterine pressure. It allowed to identify 11 contractions. FIG. 24 presents a recording of intrauterine pressure taken simultaneously to the recording of the ultrasound scan that was used in analysis presented in FIGS. 22 and 23. It allowed identifying a total of 11 contractions, just as deformable models-based method. Deformable models network appears superior to M-mode recording which did not provide significant information on uterine contractile activity. FIGS. 20 and 22 are examples of relatively high sensitivity of the M mode method to noisy signals. In this particular case, patient breathing movements caused the movement of the whole organ (the uterus) which affected the quality of an image produced using this method. As can be noted, the Snake Studio method produced the result which is possible to interpret as uterine contractions. As presented further on FIG. 24, only the recording produced by Snake Studio is comparable to the changes of intrauterine pressure. The application of the abovementioned method yielded the same number of contractions as an objective measurement of intrauterine pressure. In this case, M mode method showed to be noise sensitive and it did not produce a result which could be further analyzed.

Patient SS08

Age: 28

Fertility Profile in the early follicular phase: FSH 12.4 IU/ml; LH 2.0 IU/ml; E2 15.2 pg/ml; PRL 24 ng/ml; T 0.62 ng/ml

Stimulation protocol: short flare protocol with 0.1 mg diphereline/day+300 IU Fostimon from CD5 to 11

Ovarian response: 1 follicle growing

Endometrial response: good, endometrial thickness 10 mm

Concentration of estradiol at the end of COS: 319 pg/ml, PG 0.75 ng/ml

Comment: cycle abandoned due to insufficient ovarian response, IUP measurements conducted.

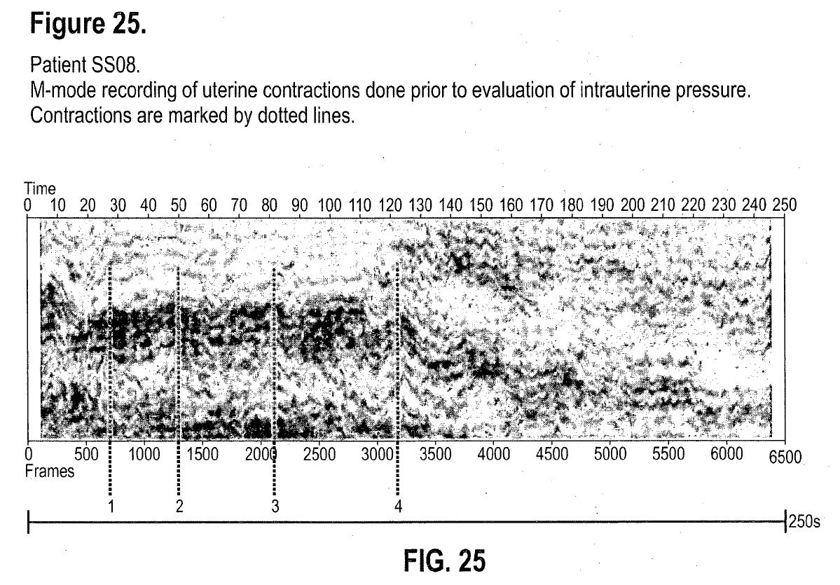

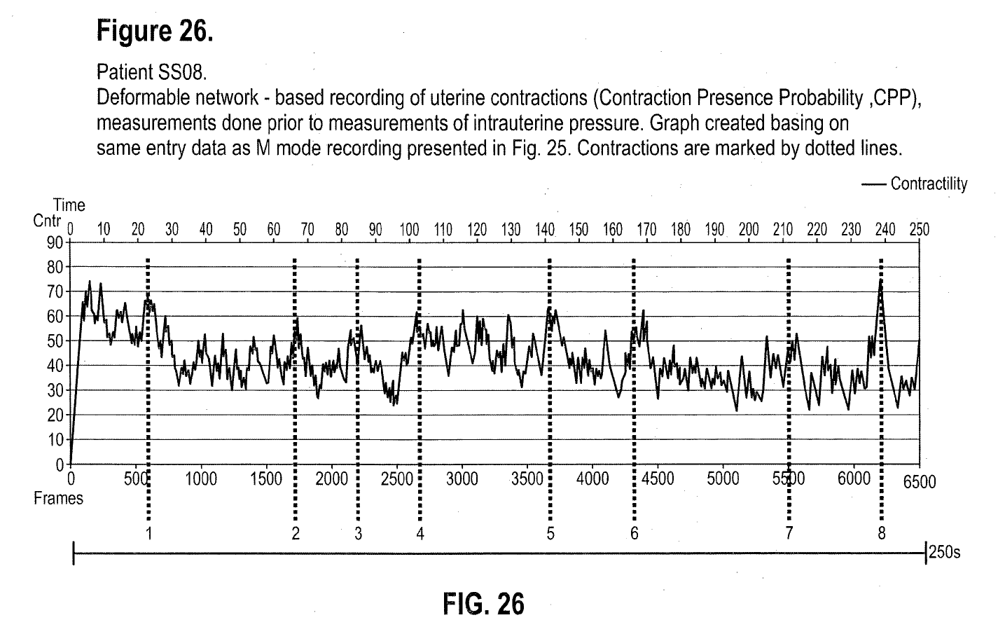

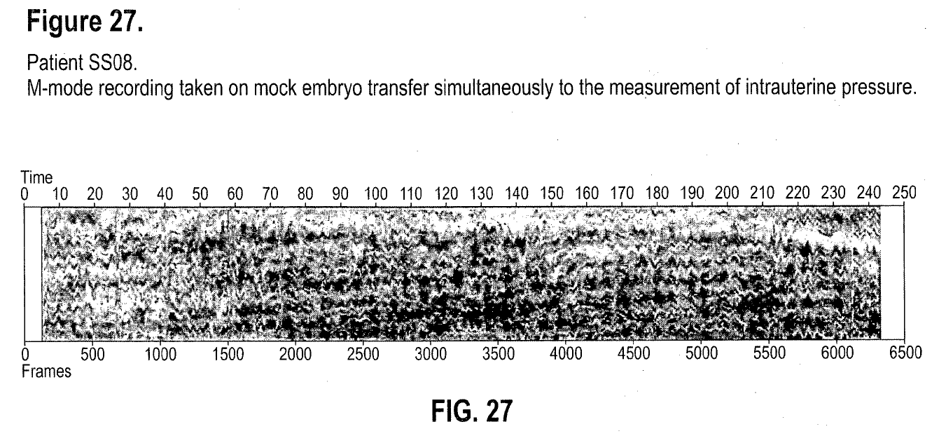

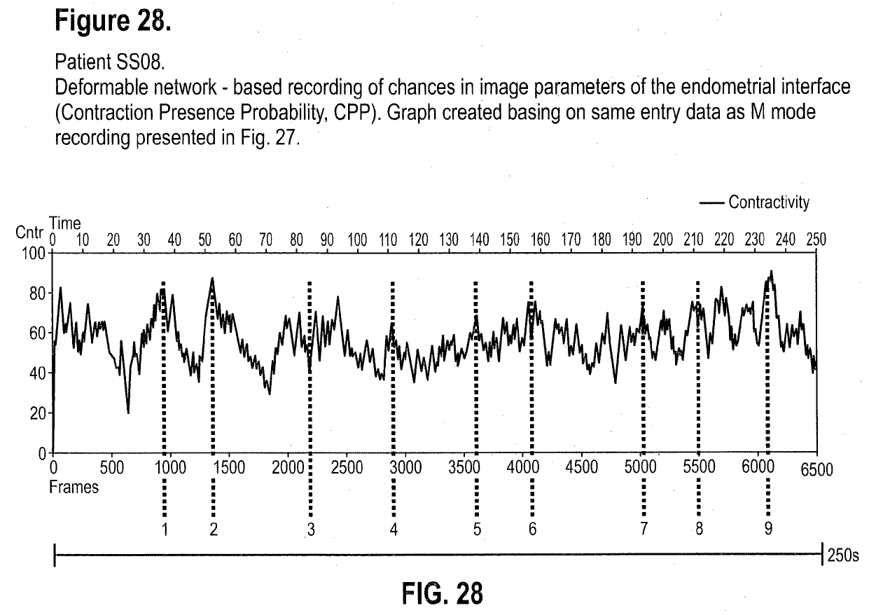

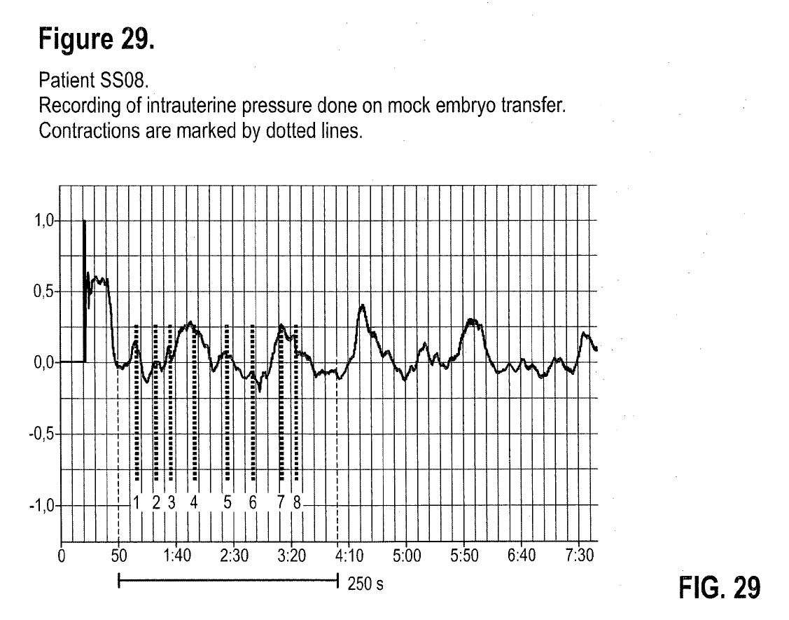

FIG. 25 presents the M-mode recording of sagittal uterine transection taken before insertion of intrauterine pressure catheter (mock embryo transfer). FIG. 26 illustrates the deformable models network based recording of changes in image parameters of the endometrial interface (Contraction Presence Probability, CPP). The graph was constructed using the same source data as presented on FIG. 25. FIG. 27 illustrates the M-mode recording taken during mock embryo transfer. FIG. 28 illustrates the deformable network-based recording of changes in image parameters of the endometrial interface measurements taken during the mock embryo transfer. FIG. 28 presents a measurement of intrauterine pressure taken during the mock embryo transfer. changes are reflected by changes of Contraction Presence Probability. In M mode recording presented on FIG. 2, it was possible to identify 4 contractions in the initial 120 seconds of recording. Further identification of contractions was not possible due to noisy signal. However, in deformable models network based method it was possible to extract more information from the same signal and identify 8 contractions. Similar number of contractions was further confirmed by measurement of intrauterine pressure (FIG. 29). Similarly, when ultrasound based evaluation of uterine contractions was performed during the mock embryo transfer, it was not possible to identify contractions in M mode. Deformable models network based method provided identification of 9 contractions. Only Snake Studio recording was comparable to changes in intrauterine pressure. The deformable models network based package provided results that are more accurate and more easily definable than those produced by M-mode recordings.

Patient SS09

Age: 42

Volunteer patient during her menstrual period, had IUP measured simultaneous with ultrasound image recording.

Additional data: Uterine contractions were identifiable while inspecting the ultrasound recording. IUP recording demonstrated intensive uterine contractile activity.

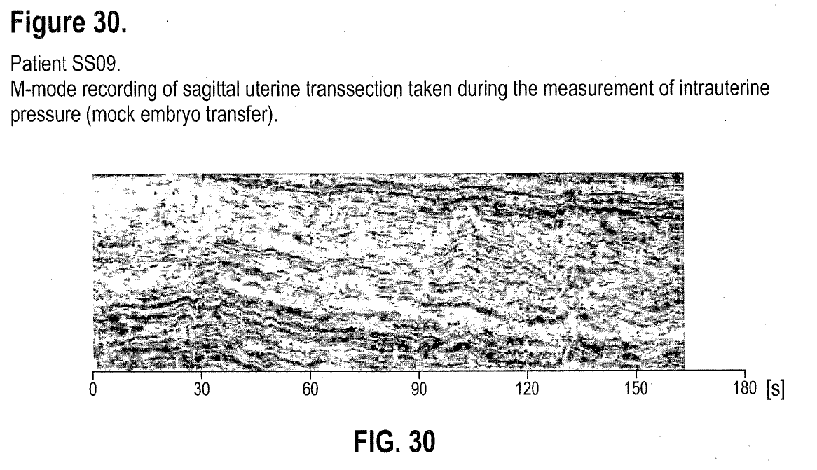

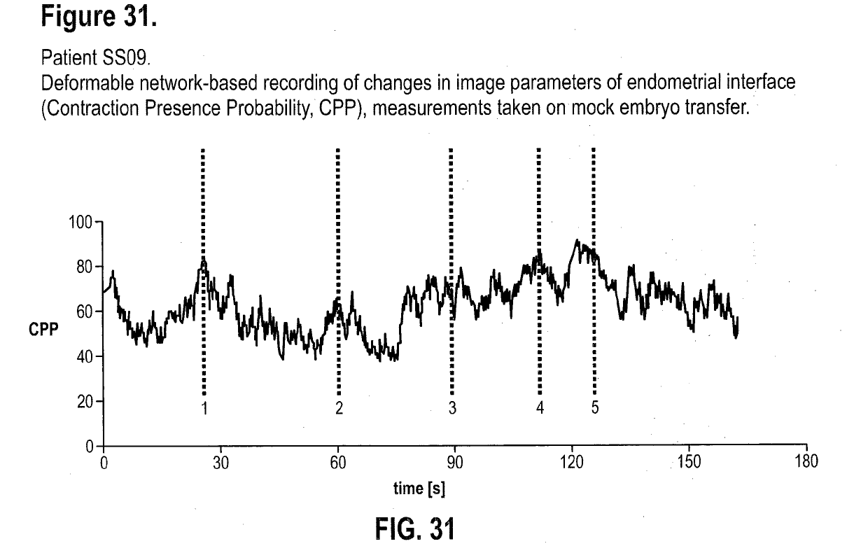

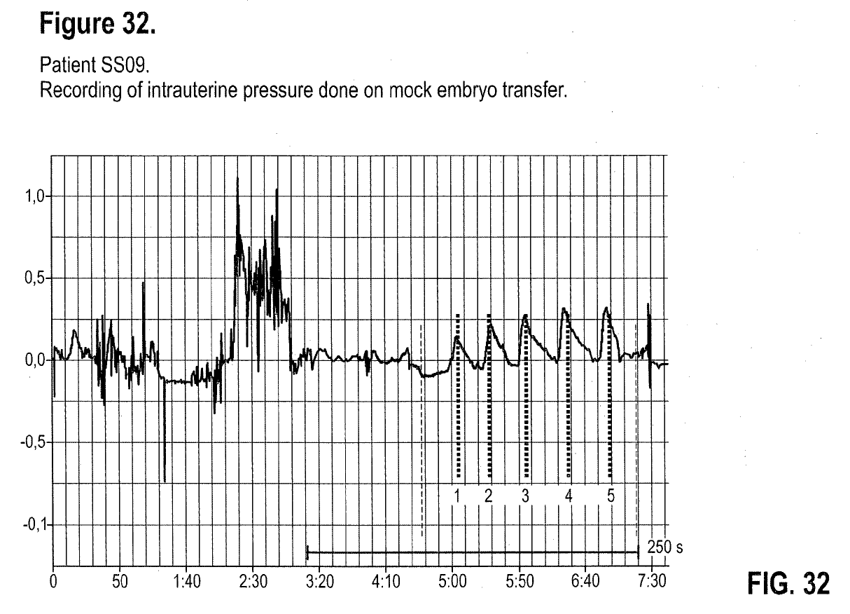

FIG. 30 illustrates the M-mode recording taken during mock embryo transfer. FIG. 31 illustrates the deformable network-based recording of changes in image parameters of the endometrial interface measurements taken during the mock embryo transfer. FIG. 32 is a comparison of recording s of intrauterine pressure (IUP) and CPP. CPP recordings done using analysis of Raw data files produced by Snake Studio. Graph Pad Prism package was used to produce the graphs of CPP changes in time. In-mode assessments are inconclusive due to lack of appropriate endometrial thickness. Snake Studio graph is significantly better in reflecting changes of intrauterine pressure.

FIG. 29 shows M mode recording of uterine contractions, which is unclear and determination of presence of any contraction is complicated/disputable. In contrast, the Snake Studio recording based on the same ultrasound sequence presented on FIG. 30 is demonstrating the visible and notable changes of CPP, representing the uterine contractions, which—as seen at FIG. 31—is better corresponding to changes in intrauterine pressure. In conclusion, for that set of data, the deformable models network based package provided results that are more accurate and more easily definable than those produced by M-mode recordings.

As described above, embodiments of the present invention provide a clear representation of uterine contractile activity. This can be used in embryo transfer procedures wherein the uterine contractile activity is controlled by administering an oxytocin antagonist. The oxytocin antagonist can be any oxytocin antagonist, such as but not limited to atosiban or barusiban. Atosiban is a marketed Ferring product in Europe (Tractocile®). Atosiban is described in European Patent No. EP 0112809, entitled Vasotocin Derivatives, incorporated herein by reference, and included in this provisional application as Attachment 1. Barusiban is described in PCT Publication Nos. WO 1998/027636 and WO 2006/121362, both of which are incorporated herein by reference, and included with this provisional patent application as Attachments 2 and 3 respectively.

Oxytocin antagonists are also used to delay pre-term birth. For example, in a method of delaying or preventing pre-term labor and birth, Atosiban is administered in three boluses, and the subject uterine imaging method could facilitate the determination of whether and when to administer the first bolus in a in pre-term labour. In an implementation of the present invention, pre-term labor is diagnosed by determining the frequency and intensity of uterine contractions as described above. Atosiban is administered to slow or stop the contractions to prevent pre-term birth. Atosiban can be administered in three doses. Atosiban can be administered in a first injection of 0.9 ml intravenous bolus over one minute with a dose of 6.75 mg, in a second injection of 24 ml/hour over three hours of intravenous loading at a dose of 18 mg/hour, and a third injection via intravenous infusion of 8 ml/hour at a dose of 6 mg/hour.

It will be appreciated that Barusiban may also be used to prevent, slow or stop pre-term uterine contractile activity.

Embodiments of the subject matter and the operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, data processing apparatus.

Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices). The operations described in this specification can be implemented as operations performed by a data processing apparatus on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones or combinations of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a smart telephone, a tablet device, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable-results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

The following references and publications are disclosed or discussed herein, and are incorporated as attachments in their entirety:
Attachment 1: European Patent No. EP 0112809, “Vasotocin Derivatives”
Attachment 2: Fanchin R. Human Reproduction 1998; 13(7):1968
Attachment 3: Lesny P, Human Reproduction 1998; 13(6):1540 Attachment 4: Handler J et al. Theriogenology 2003, 59:1381
Attachment 5: Kass M, Witkin A., and Terzopooulos, International Journal of Computer Vision; 1988; 1(4):321
Attachment 6: Liang et al., Medical Image Analysis 2006; 10(2):215-233
Attachment 7: Gunn SR and Nixon MS, IEEE Transactions on Pattern Analysis and Machine Intelligence Archive 1997; 19(1): 63.

A number of embodiments of the invention have been described. Nevertheless, it will be understood that various modifications may be made without departing from the spirit and scope of the invention. Accordingly, other embodiments are within the scope of the following claims.

What is claimed is:

1. A computer implemented method comprising:
recording ultrasound images of a subject uterus over a period of time,
analyzing the images using a deformable model network to identify uterine contractions, and
displaying uterine contractions in a graphical format.

2. The computer implemented method of claim 1 wherein: uterine contractions are determined to be within a range of 0 to 15 contractions per minute in terms frequency.

3. A method of embryo transfer comprising:
collecting of one or more eggs from a subject patient;
providing luteal support to the patient;
fertilizing the one or more eggs to provide a viable embryo;
qualifying uterine contractions in the patient by recording images of the uterine contractions and evaluating the images using a deformable models network;
reducing the level of contractions to under 4 contractions per minute;
transferring the embryo to the uterus;
continuing luteal support.

4. The method of claim 3 wherein the step of providing luteal support to the patient comprises the administration of micronized progesterone.

5. The method of claim 4 wherein the step of continuing luteal support comprises the administration of micronized progesterone.

6. The method of claim 3 wherein the step of reducing the level of uterine contractions to under 4 contractions per minute comprises administering an oxytocin antagonist.

7. The method of claim 6 wherein the oxytocin antagonist is atosiban or barusiban.

8. A computer implemented method of analyzing uterine images comprises:
recording uterine images over a period of time;
setting reference axes for use in a deformable model network;
setting the outer snake surrounding the endometrium of the subject uterus;
setting the inner snake within the endometrium of the subject uterus;
applying one or more image filters to enhance one or more features of interest;
relaxing the snakes until both meet at the endometrium perimeter;
displaying the recording and snake movement on a user display.

9. A diagnostic method for determining susceptibility of a patient to embryo transfer comprising:
measuring uterine contractile activity using a computer implemented method further comprising;
recording ultrasonic uterine images over a period of time;
setting reference axes for use in a deformable model network;
setting the outer snake surrounding the endometrium of the subject uterus;
setting the inner snake within the endometrium of the subject uterus;
applying one or more image filters to enhance one or more features of interest;
relaxing the snakes until both meet at the endometrium perimeter;
displaying the recording and snake movement on a user display identifying uterine contractile activity on the displayed recording and determining whether such contractile activity is within a minimum or maximum range for period and intensity.

10. A method of controlling uterine contractile activity comprising:
identifying the level of uterine contractile activity using the method of claim 7; and
administering an oxytocin antagonist.

11. The method of controlling uterine contractile activity of claim 10 wherein the oxytocin antagonist is atosiban or barusiban.

12. A diagnostic method for determining premature contractions in pregnant mammals comprising:
measuring uterine contractile activity using a computer implemented method further comprising;
recording ultrasonic uterine images over a period of time;
setting reference axes for use in a deformable model network;
setting the outer snake surrounding the endometrium of the subject uterus;
setting the inner snake within the endometrium of the subject uterus;
applying one or more image filters to enhance one or more features of interest;
relaxing the snakes until both meet at the endometrium perimeter;
displaying the recording and snake movement on a user display;
identifying uterine contractile activity on the displayed recording; and
determining whether such contractile activity is within a minimum or maximum range for period and intensity.

13. A method of controlling premature contractions in mammals comprising:
identifying the level of uterine contractile activity using the method of claim 10; and
administering an oxytocin antagonist.

14. The method of claim 11 wherein the oxytocin antagonist is barusiban.

15. The method of claim 11 wherein the oxytocin antagonist is atosiban.

16. The method of claim 13 wherein the atosiban is administered in three doses.

17. The method of claim 13 wherein the atosiban is administered in a first injection of 0.9 ml intravenous bolus over one minute with a dose of 6.75 mg, in a second injection of 24 ml/hour over three hours of intravenous loading at a dose of 18 mg/hour, and a third injection via intravenous infusion of 8 ml/hour at a dose of 6 mg/hour.

18. A system for analyzing uterine images comprising:
data processing apparatus configured to analyze recorded ultrasound images of a subject uterus taken over a period of time, wherein the data processing apparatus is configured to analyze the images using a deformable model network, the data processing apparatus being configured to execute the following steps:
setting reference axes for use in the deformable model network;
setting an outer snake surrounding the endometrium of the subject uterus;
setting an inner snake within the endometrium of the subject uterus;
applying one or more image filters to enhance one or more features of interest;
relaxing the snakes until both meet at the endometrium perimeter;
displaying the recording and snake movement on a user display.

19. A system for detecting uterine contractions comprising:
ultrasound apparatus for imaging the uterus;
data recording apparatus for recording ultrasonic uterine images over a period of time;
data processing apparatus configured to analyze the recorded images using a deformable model network to identify uterine contractions; and
display apparatus for displaying uterine contractions in a graphical format.

20. The system for detecting uterine contractions of claim 19, wherein the data processing apparatus is configured to execute the following steps:
setting reference axes for use in the deformable model network;
setting an outer snake surrounding the endometrium of the uterus;
setting an inner snake within the endometrium of the uterus;
applying one or more image filters to enhance one or more features of interest;
relaxing the snakes until both meet at the endometrium perimeter; and
displaying the recording and snake movement on the display apparatus.

21. A system arranged for analyzing uterine images comprising:
data processing apparatus configured to analyze recorded ultrasound images of a subject uterus taken over a period of time, wherein the data processing apparatus is configured to analyze the images using a deformable model network, the data processing apparatus being configured to execute the following steps:
setting reference axes for use in the deformable model network;
setting an outer snake surrounding the endometrium of the subject uterus;
setting an inner snake within the endometrium of the subject uterus;
applying one or more image filters to enhance one or more features of interest;
relaxing the snakes until both meet at the endometrium perimeter;
displaying the recording and snake movement on a user display.

22. A system arranged for detecting uterine contractions comprising:
ultrasound apparatus for imaging the uterus;
data recording apparatus for recording ultrasonic uterine images over a period of time;
data processing apparatus configured to analyze the recorded images using a deformable model network to identify uterine contractions; and
display apparatus for displaying uterine contractions in a graphical format.

23. The system arranged for detecting uterine contractions of claim 19, wherein the data processing apparatus is configured to execute the following steps:
setting reference axes for use in the deformable model network;
setting an outer snake surrounding the endometrium of the uterus;
setting an inner snake within the endometrium of the uterus;
applying one or more image filters to enhance one or more features of interest;
relaxing the snakes until both meet at the endometrium perimeter; and
displaying the recording and snake movement on the display apparatus.
